(19) United States
(12) Patent Application Publication
(10) Pub. No.: US 2010/0286219 A1
(43) Pub. Date: Nov. 11, 2010

(54) 1, 3-Dihydroimidazoles for Treating Cardiovascular Disorders

(75) Inventor: Patricio Manuel Vieira Araújo Soares Da Silva, Porto (PT)

Correspondence Address: CONLEY ROSE, P.C., 5601 GRANITE PARKWAY, SUITE 750, PLANO, TX, 75024, US

(73) Assignee: BIAL - PORTELA & CA S.A., S. Mamede do Coronado (PT)

(21) Appl. No.: 12/522,525

(22) PCT Filed: Jan. 10, 2008
(86) PCT No.: PCT/PT08/00001

§ 371 Date: Dec. 29, 2009

(30) Foreign Application Priority Data
Jan. 12, 2007 (GB) 0700635.6

(51) Int. Cl. A61K 31/4164; C07D 405/04; A61K 31/4178; C07D 233/84; A61P 9/00; A61P 25/06; A61P 25/22

(52) U.S. Cl. 514/392; 548/311.4; 548/324.5

(57) ABSTRACT

A method comprising utilizing a compound of formula I:

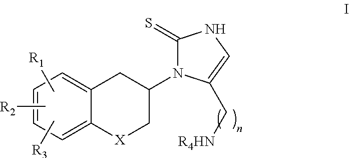

where R₁, R₂ and R₃ are the same or different and signify hydrogens, halogens, alkyl, alkylaryl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof; wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine, in the manufacture of a medicament for the treatment of one or more of the following indications: congestive heart failure, angina, arrhythmias, circulatory disorders, Raynaud's Phenomenon, migraine, and anxiety disorders.

### CROSS-REFERENCE TO RELATED APPLICATIONS

This application is a filing under 35 U.S.C. 371 of International Application No. PCT/PT2008/000001 filed Jan. 10, 2008, entitled “1,3-Dihydroimidazoles for Treating Cardiovascular Disorders,” claiming priority of Great Britain Patent Application No. 0700635.6 filed Jan. 12, 2007, which applications are incorporated by reference herein in their entirety.

### FIELD OF THE INVENTION

The present invention relates to new therapeutic applications involving the following class of compounds of formula I:

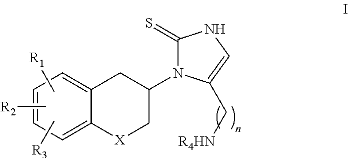

where R₁, R₂ and R₃ are the same or different and signify hydrogens, halogens, alkyl, alkylaryl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof; wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine.

### BACKGROUND OF THE INVENTION

Particular compounds of formula I include: (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5-(2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3-dihydroimidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-aminoethyl)-1-(6-hydroxythiochroman-3-yl)-1,3-dihydroimidazole-2-thione; (R,S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole7-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or (R)-1-chroman-3-yl-5-(2-methylaminoethyl)-1,3-dihydroimidazole-2-thione.

More particularly, the invention relates to drug combinations involving the following specific salts of compounds of formula I: (S)-5-(2-aminoethyl)-1-(1,2,3,4-tetrahydronaphthalen-2-yl)-1,3-dihydroimidazole-2-thione hydrochloride; (S)-5-(2-aminoethyl)-1-(5,7-difluoro-1,2,3,4-tetrahydronaphthalen-2-yl)-1,3-dihydroimidazole-2-thione hydrochloride; (R)-5-(2-aminoethyl)-1-chroman-3-yl-1,3-dihydroimidazole-2-thione hydrochloride; (R)-5-(2-aminoethyl)-1-(6-hydroxychroman-3-yl)-1,3-dihydroimidazole-2-thione hydrochloride; (R)-5-(2-aminoethyl)-1-(8-hydroxychroman-3-yl)-1,3-dihydroimidazole-2-thione hydrochloride; (R)-5-(2-aminoethyl)-1-(6-methoxychroman-3-yl)-1,3-dihydroimidazole-2-thione hydrochloride; (R)-5-(2-aminoethyl)-1-(8-methoxychroman-3-yl)-1,3-dihydroimidazole-2-thione hydrochloride; (R)-5-(2-aminoethyl)-1-(6-fluorochroman-3-yl)-1,3-dihydroimidazole-2-thione hydrochloride; (R)-5-(2-aminoethyl)-1-(8-fluorochroman-3-yl)-1,3-dihydroimidazole-2-thione hydrochloride; (R)-5-(2-aminoethyl)-1-(6,7-difluorochroman-3-yl)-1,3-dihydroimidazole-2-thione hydrochloride; (R)-5-(2-aminoethyl)-1-(6,8-difluorochroman-3-yl)-1,3-dihydroimidazole-2-thione hydrochloride; (S)-5-(2-aminoethyl)-1-(6,8-difluorochroman-3-yl)-1,3-dihydroimidazole-2-thione hydrochloride; (R)-5-(2-aminoethyl)-1-(6,7,8-trifluorochroman-3-yl)-1,3-dihydroimidazole-2-thione hydrochloride; (R)-5-(2-aminoethyl)-1-(6-chloro-8-methoxychroman-3-yl)-1,3-dihydroimidazole-2-thione hydrochloride; (R)-5-(2-aminoethyl)-1-(6-methoxy-8-chlorochroman-3-yl)-1,3-dihydroimidazole-2-thione hydrochloride; (R)-5-(2-aminoethyl)-1-(6-nitrochroman-3-yl)-1,3-dihydroimidazole-2-thione hydrochloride; (R)-5-(2-aminoethyl)-1-(8-nitrochroman-3-yl)-1,3-dihydroimidazole-2-thione hydrochloride; (R)-5-(2-aminoethyl)-1-[6-(acetylamino)chroman-3-yl]-1,3-dihydroimidazole-2-thione hydrochloride; (R)-5-aminomethyl-1-chroman-3-yl-1,3-dihydroimidazole-2-thione hydrochloride; (R)-5-aminomethyl-1-(6-hydroxychroman-3-yl)-1,3-dihydroimidazole-2-thione hydrochloride; (R)-5-(2-aminoethyl)-1-(6-hydroxy-7-benzylchroman-3-yl)-1,3-dihydroimidazole-2-thione hydrochloride; (R)-5-aminomethyl-1-(6,8-difluorochroman-3-yl)-1,3-dihydroimidazole-2-thione hydrochloride; (R)-5-(3-aminopropyl)-1-(6,8-difluorochroman-3-yl)-1,3-dihydroimidazole-2-thione hydrochloride; (S)-5-(3-aminopropyl)-1-(5,7-difluoro-1,2,3,4-tetrahydronaphthalen-2-yl)-1,3-dihydroimidazole-2-thione hydrochloride; (R,S)-5-(2-aminoethyl)-1-(6-hydroxythiochroman-3-yl)-1,3-dihydroimidazole-2-thione hydrochloride; (R,S)-5-(2-aminoethyl)-1-(6-methoxythiochroman-3-yl)-1,3-dihydroimidazole-2-thione hydrochloride; (R)-5-(2-benzylaminoethyl)-1-(6-methoxychroman-3-yl)-1,3-dihydroimidazole-2-thione hydrochloride; (R)-5-(2-benzylaminoethyl)-1-(6-hydroxychroman-3-yl)-1,3-dihydroimidazole-2-thione hydrochloride; (R)-1-(6-hydroxychroman-3-yl)-5-(2-methylaminoethyl)-1,3-dihydroimidazole-2-thione hydrochloride; (R)-1-(6,8-difluorochroman-3-yl)-5-(2-methylaminoethyl)-1,3-dihydroimidazole-2-thione hydrochloride or (R)-1-chroman-3-yl-5-(2-methylaminoethyl)-1,3-dihydroimidazole-2-thione hydrochloride.

Most particularly, the invention relates to the use of the following specific compound of formula I: (R)-5-(2-aminoethyl)-1-(6,8-difluorochroman-3-yl)-1,3-dihydroimidazole-2-thione and pharmaceutically acceptable salts thereof, especially the hydrochloride salt.

Preparation of compounds of formula I is described in WO 2004/033447.

As used herein, the term treatment and variations such as ‘treat’ or ‘treating’ refer to any regime that can benefit a human or non-human animal. The treatment may be in respect of an existing condition or may be prophylactic (preventative treatment). Treatment may include curative, alleviation or prophylactic effects. The treatment may also involve curing, alleviating or preventing symptoms associated with the disorder rather than acting on the underlying cause of the disorder. For example in the treatment of anxiety, the compounds of the present invention may cure, alleviate or prevent changes in body temperature, flushing, palpitations, etc.

### SUMMARY OF THE INVENTION

The invention relates to the following therapeutic applications of these compounds: treatment of congestive heart failure, treatment of angina, treatment of arrhythmias, treatment of circulatory disorders such as Raynaud's Phenomenon (sometimes known as “Raynaud's Disease”), treatment of migraine, and treatment of anxiety and anxiety disorders.

The invention includes the use of a compound of formula I or a pharmaceutically acceptable salt thereof in the manufacture of a medicament for treating congestive heart failure.

The invention includes the use of a compound of formula I or a pharmaceutically acceptable salt thereof in the manufacture of a medicament for treating angina.

The invention includes the use of a compound of formula I or a pharmaceutically acceptable salt thereof in the manufacture of a medicament for treating arrhythmias.

The invention includes the use of a compound of formula I or a pharmaceutically acceptable salt thereof in the manufacture of a medicament for treating circulatory disorders such as Raynaud's Phenomenon (sometimes known as “Raynaud's Disease”).

The invention includes the use of a compound of formula I or a pharmaceutically acceptable salt thereof in the manufacture of a medicament for treating migraine.

The invention includes the use of a compound of formula I or a pharmaceutically acceptable salt thereof in the manufacture of a medicament for treating anxiety disorders.

The invention also includes a method of treating one or more of the conditions mentioned above comprising administering a therapeutically effective amount of a compound of formula I or a pharmaceutically acceptable salt thereof to a subject in need thereof.

The compounds of formula I or their pharmaceutically acceptable salts may be formulated into a pharmaceutical composition.

The composition may further comprise another active pharmaceutical ingredient. Suitable active ingredients are described in PCT/PT2007/000002.

The composition may also comprise suitable a pharmaceutically acceptable excipient and/or pharmaceutically acceptable carrier.

### BRIEF DESCRIPTION OF THE DRAWINGS

Reference is now made to the accompanying drawings, in which:

FIG. 1 is a graph showing the time-dependent decrease of noradrenaline levels in the left ventricle of mice treated orally with 100 mg/kg of 4 or nepicastat 1. Symbols are means of 5 determinations per group; vertical lines indicate S.E.M.

FIG. 2 is two graphs showing noradrenaline levels in the mouse left ventricle and brain parietal cortex 9 h after oral administration of 4 or nepicastat 1. Symbols are means of 5 determinations per group; vertical lines indicate S.E.M.

FIG. 3 is four graphs showing noradrenaline levels in the rat heart (left atrium and left ventricle) and brain (frontal and parietal cortex) 9 h after the oral administration of 4 or nepicastat 1. Columns are means of 5 determinations per group; vertical lines indicated S.E.M.

### DETAILED DESCRIPTION

### Results

### In Vitro Studies

Incubation of SK-N-SH cells in the presence of increasing concentrations of dopamine resulted in a concentration-dependent formation of noradrenaline, yielding Km (in μM) and Vmax (in nmol mg protein-1 h-1) values of 20.6±1.6 and 153.8±4.4, respectively. From these kinetic parameters, a concentration of dopamine approaching saturation (50 mM) was chosen for use in inhibition studies. As listed in Table 1 compounds 2, 3, 4, 5, 6, 7, 8, 10, 12, 16, 19, 24, 26, 28 and 29 were found to markedly inhibit DβH activity. Compounds 2, 3, 4 and nepicastat 1 (the reference compound) produced a concentration-dependent decrease in the β-hydroxylation of dopamine with IC50 values in the low nM range against human DβH activity (see Table 2). Compound 4 was chosen for further in vivo studies, being the compound most closely related to nepicastat 1 in order to provide conclusive evidence that the structural modifications made to the molecule as part of the present invention are responsible for the surprisingly markedly improved biological properties observed.

TABLE 1
Effect of selected compounds (5 μM)
on DβH activity in SK-N-SH cells.

| No. | Mean ± SEM |
|---|---|
| 1 | 0.0 ± 0.3 |
| 2 | 1.6 ± 0.3 |
| 3 | 4.1 ± 0.6 |
| 4 | 3.3 ± 0.3 |
| 5 | 8.1 ± 0.3 |
| 6 | 6.9 ± 0.6 |
| 7 | 8.0 ± 0.1 |
| 8 | 9.4 ± 0.7 |
| 9 | 50.2 ± 1.9 |
| 10 | 8.2 ± 0.7 |
| 11 | 36.7 ± 4.4 |
| 12 | 3.0 ± 0.5 |
| 13 | 94.0 ± 3.1 |
| 14 | 77.9 ± 2.2 |
| 15 | 86.1 ± 2.7 |
| 16 | 0.0 ± 0.6 |
| 17 | 53.2 ± 3.9 |
| 18 | 94.8 ± 1.2 |
| 19 | 6.9 ± 0.5 |
| 20 | 16.8 ± 4.8 |
| 21 | 124.8 ± 6.5 |
| 22 | 17.8 ± 2.1 |
| 23 | 54.5 ± 9.9 |
| 24 | 0.0 ± 1.9 |
| 25 | 66.0 ± 4.5 |
| 26 | 4.5 ± 1.9 |
| 27 | 15.5 ± 5.8 |
| 28 | 2.6 ± 1.6 |
| 29 | 2.2 ± 2.5 |
| 30 | 99.4 ± 2.8 |
| 31 | 27.3 ± 0.4 |

Values are quoted as % of control.

TABLE 2
IC₅₀ values (in nM) for inhibition of DβH in SK-N-SH cells.

| Compound | IC₅₀ (in nM) |
|---|---|
| 2 | 60 (14, 250) |
| 3 | 91 (56, 147) |
| 4 | 105 (69, 161) |
| Nepicastat 1 | 36 (28, 46) |

### In Vivo Studies

### Mouse

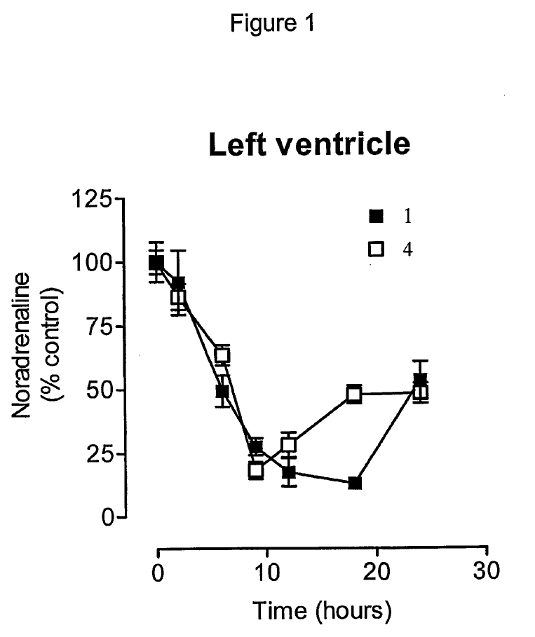

The time course experiments for compound 4 and nepicastat (1) in the heart at 100 mg/kg suggests that both compounds are long acting. Time of maximum effect (Tmax) for noradrenaline tissue reduction by both 4 and 1 appears to be at 9 h post-dose (FIG. 1). Thereafter, noradrenaline tissue levels recover, reaching 50% recovery of initial tissue levels at 24 h.

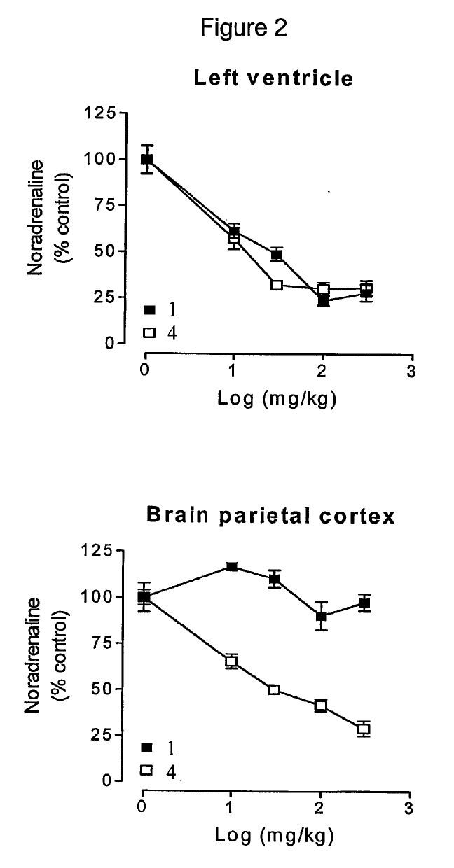

At Tmax (9 h after administration), both 4 and 1 reduced noradrenaline levels in a dose-dependent manner in left ventricle. For both 4 and 1, the maximal inhibitory effect was attained at a dose of 100 mg/kg. In contrast to that found in the heart, 4 failed to affect noradrenaline tissue levels in the brain parietal cortex, whereas 1 produced a dose-dependent decrease in noradrenaline levels in this area of the brain (FIG. 2).

### Rat

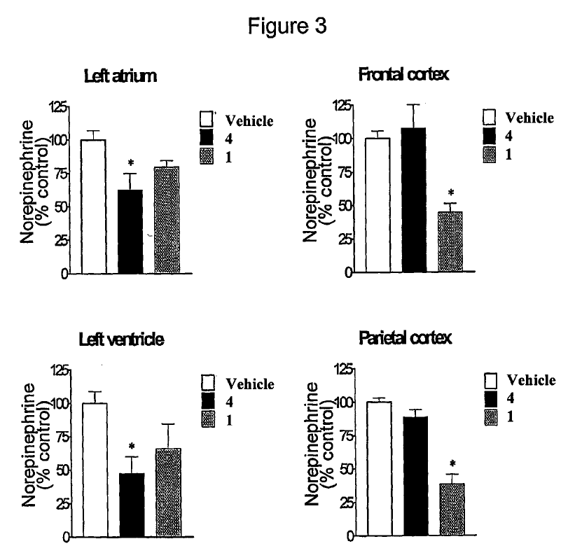

As shown in the mouse, the effects of both 4 and 1 upon noradrenaline were dependent on the dose administered and reached its maximum at 9 h (data not shown). However, as depicted in FIG. 3, the inhibitory effects of 4 (100 mg/kg) upon noradrenaline levels in both the left atrium and the left ventricle were more pronounced than those elicited by 1 (100 mg/kg). Again, as observed in the mouse, 4 failed to affect noradrenaline tissue levels in the brain parietal cortex and the brain frontal cortex, whereas 1 produced a marked decrease in noradrenaline levels in these brain areas.

It is concluded that 4, in stark contrast to nepicastat 1, exerts its inhibitory effects upon DβH exclusively in the periphery, being devoid of inhibitory effects in the brain.

### Conclusion

Some compounds of general formula I are very potent dopamine-β-hydroxylase inhibitors and have potentially valuable pharmaceutical properties in the treatment of some cardiovascular disorders, where a reduction in the enzymatic hydroxylation of dopamine to noradrenaline may be of therapeutic benefit, such as hypertension and chronic heart failure. The possibility to use a long-acting DβH inhibitor with limited access to the brain (CNS), such as compound 4 opens new perspectives in the treatment of hypertension and chronic heart failure by improving potency and selectivity of DβH inhibition in the periphery.

The invention disclosed herein is exemplified by the following examples of preparation, which should not be construed to limit the scope of the disclosure. Alternative pathways and analogous structures may be apparent to those skilled in the art.

### EXAMPLES

### Example 1

### (R)-5-aminomethyl-1-(6,8-difluorochroman-3-yl)-1,3-dihydroimidazole-2-thione hydrochloride compound 3, table 1)

A stirred mixture of (R)-6,8-difluorochroman-3-ylamine hydrochloride (0.22 g, 1.0 mmol), [3-(tert-butyldimethylsilanyloxy)-2-oxopropyl]carbamic acid tert-butyl ester (0.33 g, 1.1 mmol), potassium thiocyanate (0.11 g, 1.1 mmol) and acetic acid (0.3 mL, 5.0 mmol) in ethyl acetate (3 mL) was refluxed for 2 hours, cooled to room temperature, then washed by sodium bicarbonate solution, dried over anhydrous magnesium sulphate and evaporated in vacuo. The residue was purified by the column chromatography over silica gel using ethyl acetate-petroleum ether mixture as eluent. The resulting oil (0.23 g) was dissolved in ethyl acetate (2 ml), whereupon 2M HCl solution in ethyl acetate was added (2 mL, 4 mmol) and the mixture was stirred for 2 hours at room temperature. The precipitate was removed by filtration and washed with ethyl acetate to give crystals of m.p. 192° C. (decomp.).

### Examples 2-3

By the application of the above described technique and related procedures known to those skilled in the art and using the appropriate chroman-3-ylamines hydrochlorides, the following compounds were prepared:
(R)-5-aminomethyl-1-chroman-3-yl-1,3-dihydroimidazole-2-thione hydrochloride (compound 24, table 1)
(R)-5-aminomethyl-1-(6-hydroxychroman-3-yl)-1,3-dihydroimidazole-2-thione hydrochloride (compound 22, table 1).

### Example 4

### (R,S)-5-aminomethyl-1-(6-hydroxythiochroman-3-yl)-1,3-dihydroimidazole-2-thione hydrochloride

A stirred mixture of 6-hydroxythiochroman-3-ylamine hydrochloride (0.22 g, 1.0 mmol), [3-(tert-butyldimethylsilanyloxy)-2-oxopropyl]carbamic acid tert-butyl ester (0.33 g, 1.1 mmol), potassium thiocyanate (0.11 g, 1.1 mmol) and acetic acid (0.3 mL, 5.0 mmol) in ethyl acetate (3 mL) was refluxed for 2 hours, then cooled to room temperature, and washed by sodium bicarbonate solution, dried over anhydrous magnesium sulphate and evaporated in vacuo. The residue was purified by column chromatography on silica using ethyl acetate-petroleum ether mixture as eluent. The resulting oil (0.25 g) was dissolved in ethyl acetate (2 ml), whereupon 2M HCl solution in ethyl acetate was added (2 mL, 4 mmol) and the mixture was stirred for 2 hours at room temperature. The precipitate was removed by filtration and washed with ethyl acetate to give crystals, which decomposed without melting.

### Example 5

### (3,4-Dihydroxybutyl)carbamic acid tert-butyl ester

To a stirred solution of 4-amino-1,2-propanediol (2.10 g, 20 mmol) in ethanol (50 mL) at room temperature was added di-tert-butyldicarbonate (4.80 g, 22 mmol) in one portion. The resulting mixture was stirred at room temperature for two hours, then evaporated in vacuo and purified by column chromatography on silica using ethyl acetate-petroleum ether mixture as eluent to afford colourless oil.

### Examples 6-7

By the application of the above described technique and related procedures known to those skilled in the art and using the appropriate N-substituted 4-amino-1,2-propanediols, the following compounds were prepared:
(3,4-Dihydroxybutyl)methylcarbamic acid tert-butyl ester
(3,4-Dihydroxybutyl)benzylcarbamic acid tert-butyl ester.

### Example 8

### [4-(tert-butyldimethylsilanyloxy)-3-hydroxybutyl]carbamic acid tert-butyl ester

To a stirred solution of (3,4-dihydroxybutyl)carbamic acid tert-butyl ester (2.60 g, 12.7 mmol), triethylamine (2.03 mL, 14.50 mmol) and 4-(dimethylamino)pyridine (0.05 g, 0.4 mmol) in anhydrous dichloromethane (40 mL) at room temperature was added tert-butyldimethylchlorosilane (2.0 g, 13.17 mmol) in one portion. The resulting mixture was stirred at room temperature for 18 hours, washed with water, brine and dried over anhydrous magnesium sulfate. Filtration and concentration in vacuo gave an oil which was purified by column chromatography on silica using ethyl acetate-petroleum ether mixture as eluent to afford a colourless oil.

### Examples 9-10

By the application of the above described technique and related procedures known to those skilled in the art and using compounds from examples 6 and 7, the following compounds were prepared:
[4-(tert-butyldimethylsilanyloxy)-3-hydroxybutyl]methylcarbamic acid tert-butyl ester
[4-(tert-butyldimethylsilanyloxy)-3-hydroxybutyl]benzylcarbamic acid tert-butyl ester.

### Example 11

### [4-(tert-butyldimethylsilanyloxy)-3-oxobutyl]carbamic acid tert-butyl ester

To a solution of Dess-Martin periodinane (5.0 g, 11.8 mmol) in anhydrous dichloromethane (35 mL) at room temperature was added a solution of [4-(tert-butyldimethylsilanyloxy)-3-hydroxybutyl]carbamic acid tert-butyl ester (3.77 g, 11.8 mmol) in anhydrous dichloromethane. The resulting mixture was stirred at room temperature for one hour, evaporated in vacuo to one third of the initial volume and applied to a column packed with silica. Elution with ethyl acetate-petroleum ether solvent mixture afforded a colourless oil.

### Examples 12-13

By the application of the above described technique and related procedures known to those skilled in the art and using compounds from examples 9 and 10, the following compounds were prepared:
[4-(tert-butyldimethylsilanyloxy)-3-oxobutyl]methylcarbamic acid tert-butyl ester
[4-(tert-butyldimethylsilanyloxy)-3-oxobutyl]benzylcarbamic acid tert-butyl ester.

### Example 14

### (S)-5-(2-aminoethyl)-1-(5,7-difluoro-1,2,3,4-tetrahydronaphthalen-2-yl)-1,3-dihydroimidazole-2-thione hydrochloride, compound 2, table 1)

A stirred mixture of (S)-5,7-difluoro-1,2,3,4-tetrahydronaphthalen-2-yl amine hydrochloride (0.17 g, 0.79 mmol), [4-(tert-butyldimethylsilanyloxy)-3-oxobutyl]carbamic acid tert-butyl ester (0.28 g, 0.87 mmol), potassium thiocyanate (0.085 g, 0.85 mmol), water (0.014 mL, 0.80 mmol) and acetic acid (0.2 mL, 3.3 mmol) in ethyl acetate (2 mL) was refluxed for 7 hours, cooled to the room temperature, washed by sodium bicarbonate solution and dried over anhydrous magnesium sulphate and evaporated in vacuo. The residue was purified by column chromatography on silica using ethyl acetate-petroleum ether mixture as eluent. The resulting oil (0.24 g) was dissolved in ethyl acetate (2 ml), 2M HCl solution in ethyl acetate was added (2 mL, 4 mmol) and the mixture was stirred for 2 hours at room temperature. The precipitate was removed by filtration and washed with ethyl acetate to give crystals, which decomposed without melting.

### Example 15

By the application of the above described technique and related procedures known to those skilled in the art and using the appropriate 1,2,3,4-tetrahydronaphthalen-2-ylamines hydrochlorides, the following compounds were prepared:
(S)-5-(2-aminoethyl)-1-(1,2,3,4-tetrahydronaphthalen-2-yl)-1,3-dihydroimidazole-2-thione hydrochloride (compound 20, table 1).

### Example 16

### (R)-5-(2-aminoethyl)-1-(6,8-difluorochroman-3-yl)-1,3-dihydroimidazole-2-thione hydrochloride (compound 4, table 1)

A stirred mixture of (R)-6,8-difluorochroman-3-ylamine hydrochloride (1.68 g, 7.58 mmol), [4-(tert-butyldimethylsilanyloxy)-3-oxobutyl]carbamic acid tert-butyl ester (3.13 g, 9.85 mmol), potassium thiocyanate (0.96 g, 9.85 mmol), water (0.18 mL, 10 mmol) and acetic acid (3.0 mL, 50 mmol) in ethyl acetate (30 mL) was refluxed for 7 hours, cooled to room temperature, washed by sodium bicarbonate solution, dried over anhydrous magnesium sulphate and evaporated in vacuo. The residue was purified by column chromatography on silica using ethyl acetate-petroleum ether mixture as eluent. The resulting oil (2.15 g) was dissolved in ethyl acetate (20 ml), 2M HCl solution in ethyl acetate was added (20 mL, 40 mmol) and the mixture was stirred for 2 hours at room temperature. The precipitate was removed by filtration and washed with ethyl acetate to give crystals, which decomposed without melting.

### Examples 17-37

By the application of the above described technique and related procedures known to those skilled in the art and using the appropriate chroman-3-ylamine hydrochlorides and [4-(tert-butyldimethylsilanyloxy)-3-oxobutyl]carbamic acid tert-butyl esters, the following compounds were prepared:
(R)-5-(2-aminoethyl)-1-chroman-3-yl-1,3-dihydroimidazole-2-thione hydrochloride (compound 12, table 1)
(R)-5-(2-aminoethyl)-1-(6-hydroxychroman-3-yl)-1,3-dihydroimidazole-2-thione hydrochloride (compound 16, table 1)
(R)-5-(2-aminoethyl)-1-(8-hydroxychroman-3-yl)-1,3-dihydroimidazole-2-thione hydrochloride (compound 21, table 1)
(R)-5-(2-aminoethyl)-1-(6-methoxychroman-3-yl)-1,3-dihydroimidazole-2-thione hydrochloride (compound 23, table 1)
(R)-5-(2-aminoethyl)-1-(8-methoxychroman-3-yl)-1,3-dihydroimidazole-2-thione hydrochloride (compound 19, table 1)
(R)-5-(2-aminoethyl)-1-(6-fluorochroman-3-yl)-1,3-dihydroimidazole-2-thione hydrochloride (compound 7, table 1)
(R)-5-(2-aminoethyl)-1-(8-fluorochroman-3-yl)-1,3-dihydroimidazole-2-thione hydrochloride (compound 6, table 1)
(R)-5-(2-aminoethyl)-1-(6,7-difluorochroman-3-yl)-1,3-dihydroimidazole-2-thione hydrochloride (compound 8, table 1)
(S)-5-(2-aminoethyl)-1-(6,8-difluorochroman-3-yl)-1,3-dihydroimidazole-2-thione hydrochloride (compound 9, table 1)
(R)-5-(2-aminoethyl)-1-(6,7,8-trifluorochroman-3-yl)-1,3-dihydroimidazole-2-thione hydrochloride (compound 10, table 1)
(R)-5-(2-aminoethyl)-1-(6-chloro-8-methoxychroman-3-yl)-1,3-dihydroimidazole-2-thione hydrochloride (compound 11, table 1)
(R)-5-(2-aminoethyl)-1-(6-methoxy-8-chlorochroman-3-yl)-1,3-dihydroimidazole-2-thione hydrochloride (compound 13, table 1)
(R)-5-(2-aminoethyl)-1-(6-nitrochroman-3-yl)-1,3-dihydroimidazole-2-thione hydrochloride
(compound 18, table 1)
(R)-5-(2-aminoethyl)-1-(8-nitrochroman-3-yl)-1,3-dihydroimidazole-2-thione hydrochloride
(compound 17, table 1)
(R)-5-(2-aminoethyl)-1-[6-(acetylamino)chroman-3-yl]-1,3-dihydroimidazole-2-thione hydrochloride (compound 14, table 1)
(R)-5-(2-aminoethyl)-1-(6-hydroxy-7-benzylchroman-3-yl)-1,3-dihydroimidazole-2-thione hydrochloride (compound 15, table 1)
(R)-5-(2-Benzylaminoethyl)-1-(6-methoxychroman-3-yl)-1,3-dihydroimidazole-2-thione hydrochloride (compound 25, table 1)
(R)-5-(2-Benzylaminoethyl)-1-(6-hydroxychroman-3-yl)-1,3-dihydroimidazole-2-thione hydrochloride (compound 26, table 1)
(R)-1-(6-Hydroxychroman-3-yl)-5-(2-methylaminoethyl)-1,3-dihydroimidazole-2-thione hydrochloride (compound 27, table 1)
(R)-1-(6,8-Difluorochroman-3-yl)-5-(2-methylaminoethyl)-1,3-dihydroimidazole-2-thione hydrochloride (compound 28, table 1)
(R)-1-Chroman-3-yl-5-(2-methylaminoethyl)-1,3-dihydroimidazole-2-thione hydrochloride (compound 29, table 1).

### Example 38

### (R,S)-5-(2-aminoethyl)-1-(6-methoxythiochroman-3-yl)-1,3-dihydroimidazole-2-thione hydrochloride (compound 30, table 1)

A stirred mixture of 6-methoxythiochroman-3-ylamine hydrochloride (0.12 g, 0.50 mmol), [3-(tert-butyldimethylsilanyloxy)-2-oxopropyl]carbamic acid tert-butyl ester (0.17 g, 0.55 mmol), potassium thiocyanate (0.055 g, 0.55 mmol), water (0.009 g, 0.50 mmol) and acetic acid (0.2 mL, 3.3 mmol) in ethyl acetate (2 mL) was refluxed for 7 hours, cooled to room temperature, washed by sodium bicarbonate solution, dried over anhydrous magnesium sulphate and evaporated in vacuo. The residue was purified by column chromatography on silica using ethyl acetate-petroleum ether mixture as eluent. The resulting oil (0.12 g) was dissolved in ethyl acetate (1 ml), 2M HCl solution in ethyl acetate was added (1 mL, 2 mmol) and the mixture was stirred for 2 hours at room temperature. The precipitate was removed by filtration and washed with ethyl acetate to give crystals which decomposed without melting.

### Example 39

By the application of the above described technique and related procedures known to those skilled in the art and using the appropriate chroman-3-ylamine hydrochlorides, the following compounds were prepared: (R,S)-5-(2-aminoethyl)-1-(6-hydroxythiochroman-3-yl)-1,3-dihydroimidazole-2-thione hydrochloride (compound 31, table 1).

### Example 40

### 2-[3-(2,2-Dimethyl[1,3]dioxolan-4-yl)propyl]isoindole-1,3-dione

To a stirred solution of 3-(2,2-dimethyl-[1,3]dioxolan-4-yl)propylamine (1.05 g, 6.60 mmol) and carboethoxyphthalimide (1.45 g, 6.60 mmol) in acetonitrile (10 mL) at room temperature was added triethylamine (0.92 mL, 6.60 mmol) in one portion and the resulting mixture was stirred at room temperature for 18 hours, evaporated in vacuo and the residue was dissolved in ethyl acetate (50 mL). The solution was washed with brine, 10% citric acid solution and brine, then dried over anhydrous magnesium sulfate. Filtration and concentration in vacuo gave an oil which was purified by column chromatography on silica using ethyl acetate-petroleum ether mixture as eluent to afford a colourless oil.

### Example 41

### 2-(4,5-Dihydroxypentyl)isoindole-1,3-dione

To a stirred solution of 2-[3-(2,2-dimethyl[1,3]dioxolan-4-yl)propyl]isoindole-1,3-dione (1.65 g, 5.70 mmol) in THF (20 mL) at room temperature was added 2N HCl solution (15 mL, 30 mmol) in one portion and the resulting mixture was stirred at room temperature for two hours and then evaporated in vacuo to half of the initial volume. The residue was saturated with NaCl and extracted with ethyl acetate. The organic phase was dried by anhydrous magnesium sulfate. Filtration and concentration in vacuo afforded a colourless oil.

### Example 42

By the application of the technique described in example 8 to 2-(4,5-dihydroxypentyl)isoindole-1,3-dione, the following compound was prepared:
2-[5-(tert-Butyldimethylsilanyloxy)-4-hydroxypentyl]isoindole-1,3-dione.

### Example 43

By the application of the technique described in example 11 to 2-[5-(tert-butyldimethylsilanyloxy)-4-hydroxypentyl]isoindole-1,3-dione, the following compound was prepared:
2-[5-(tert-Butyldimethylsilanyloxy)-4-oxopentyl]isoindole-1,3-dione.

### Example 44

### (S)-5-(3-aminopropyl)-1-(5,7-difluoro-1,2,3,4-tetrahydronaphthalen-2-yl)-1,3-dihydroimidazole-2-thione hydrochloride (compound 5, table 1)

A stirred mixture of (S)-5,7-difluoro-1,2,3,4-tetrahydronaphthalen-2-yl amine hydrochloride (0.22 g, 1.0 mmol), 2-[5-(tert-butyldimethylsilanyloxy)-4-oxopentyl]isoindole-1,3-dione (0.38 g, 1.05 mmol), potassium thiocyanate (0.11 g, 1.10 mmol), water (0.18 g, 1.0 mmol) and acetic acid (0.3 mL, 5.0 mmol) in ethyl acetate (3 mL) was refluxed for 7 hours, cooled to room temperature, washed by sodium bicarbonate solution, dried over anhydrous magnesium sulphate and evaporated in vacuo. The residue was purified by column chromatography on silica using ethyl acetate-petroleum ether mixture as eluent. The resulting oil (0.18 g) was dissolved in a mixture of isopropanol (5 mL) and THF (2 mL). Water (0.8 mL) and sodium borohydride (0.066 g, 1.74 mmol) were added at room temperature and the mixture was stirred for 1.5 hours. Acetic acid (0.6 ml, 10 mmol) was added and the solution was refluxed for two hours then evaporated in vacuo to dryness. The residue was taken up into acetone, the solid was filtered off, and the filtrate was acidified with 2N HCl solution in ethyl acetate. The precipitate was collected and washed with acetone to afford crystals, which decomposed without melting.

### Example 45

### (R)-5-(3-aminopropyl)-1-(6,8-difluorochroman-3-yl)-1,3-dihydroimidazole-2-thione hydrochloride

A stirred mixture of (R)-6,8-difluorochroman-3-ylamine hydrochloride (0.11 g, 0.50 mmol), 2-[5-(tert-Butyldimethylsilanyloxy)-4-oxopentyl]isoindole-1,3-dione (0.19 g, 0.55 mmol), potassium thiocyanate (0.055 g, 0.55 mmol), water (0.009 g, 0.50 mmol) and acetic acid (0.15 mL, 2.5 mmol) in ethyl acetate (1.5 mL) was refluxed for 7 hours, cooled to the room temperature, washed by sodium bicarbonate solution, dried over anhydrous magnesium sulphate and evaporated in vacuo. The residue was purified by column chromatography on silica using ethyl acetate-petroleum ether mixture as eluent. The resulting oil (0.10 g) was dissolved in the mixture of isopropanol (2.5 mL) and THF (1 mL). Water (0.4 mL) and sodium borohydride (0.038 g, 1.0 mmol) were added at room temperature and the mixture was stirred for 1.5 hours. Acetic acid (0.3 ml, 5 mmoj) was added and the solution was refluxed for two hours and evaporated in vacuo to dryness. The residue was taken up in acetone, the solid was filtered off, and the filtrate was acidified with 2N HCl solution in ethyl acetate. The precipitate was collected and washed with acetone to afford crystals, which decomposed without melting.

### Example 46

### (R,S)-5-(3-aminopropyl)-1-(6-hydroxythiochroman-3-yl)-1,3-dihydroimidazole-2-thione hydrochloride

A stirred mixture of 6-hydroxythiochroman-3-ylamine hydrochloride (0.22 g, 1.0 mmol), 2-[5-(tert-Butyldimethylsilanyloxy)-4-oxopentyl]isoindole-1,3-dione (0.38 g, 1.05 mmol), potassium thiocyanate (0.11 g, 1.10 mmol), water (0.18 g, 1.0 mmol) and acetic acid (0.3 mL, 5.0 mmol) in ethyl acetate (3 mL) was refluxed for 7 hours, cooled to room temperature, washed by sodium bicarbonate solution, dried over anhydrous magnesium sulphate and evaporated in vacuo. The residue was purified by column chromatography on silica using ethyl acetate-petroleum ether mixture as eluent. The resulting oil (0.17 g) was dissolved in the mixture of isopropanol (5 mL) and THF (2 mL). Water (0.8 mL) and sodium borohydride (0.066 g, 1.74 mmol) were added at room temperature and the mixture was stirred for 1.5 hours. Acetic acid (0.6 ml, 10 mmoj) was added and the solution was refluxed for two hours and evaporated in vacuo to dryness. The residue was taken up into acetone, the solid was filtered off and the filtrate was acidified with 2N HCl solution in ethyl acetate. The precipitate was collected and washed with acetone to afford crystals, which decomposed without melting.

It will be clear to the person skilled in the field that minor modifications may be made to the invention as described herein without departing from the scope of the claims. Such modifications would be in the field of knowledge of the skilled person.

1. A method comprising utilizing a compound of formula I: 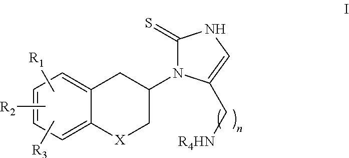
where R₁, R₂ and R₃ are the same or different and signify hydrogens, halogens, alkyl, alkylaryl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof; wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine, in the manufacture of a medicament for the treatment of one or more of the following indications: congestive heart failure, angina, arrhythmias, circulatory disorders, Raynaud's Phenomenon, migraine, and anxiety and anxiety disorders.

2. The method according to claim 1, wherein the medicament is for the treatment of congestive heart failure.

3. The method according to claim 1, wherein the medicament is for the treatment of angina.

4. The method according to claim 1, wherein the medicament is for the treatment of arrhythmias.

5. The method according to claim 1, wherein the medicament is for the treatment of circulatory disorders such as Raynaud's Phenomenon.

6. The method according to claim 1, wherein the medicament is for the treatment of migraine.

7. The method according to claim 1, wherein the medicament is for the treatment of anxiety and anxiety disorders.

8. The method according to any preceding claim 1, wherein the medicament further comprises at least one other active pharmaceutical ingredient.

9. The method according to claim 1, wherein the compound of formula I is (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5 (2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3-dihydroimidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-aminoethyl)-1-(6-hydroxythiochroman-3-yl)-1,3-dihydroimidazole-2-thione; (R,S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or (R)-1-chroman-3-yl-5-(2-methylaminoethyl)-1,3-dihydroimidazole-2-thione.

10. The method according to claim 1, wherein the pharmaceutically acceptable salt used in the manufacture of the medicament is (S)-5-(2-aminoethyl)-1-(1,2,3,4-tetrahydronaphthalen-2-yl)-1,3-dihydroimidazole-2-thione hydrochloride; (S)-5-(2-aminoethyl)-1-(5,7-difluoro-1,2,3,4-tetrahydronaphthalen-2-yl)-1,3-dihydroimidazole-2-thione hydrochloride; (R)-5-(2-aminoethyl)-1-chroman-3-yl-1,3-dihydroimidazole-2-thione hydrochloride; (R)-5-(2-aminoethyl)-1-(6-hydroxychroman-3-yl)-1,3-dihydroimidazole-2-thione hydrochloride; (R)-5-(2-aminoethyl)-1-(8-hydroxychroman-3-yl)-1,3-dihydroimidazole-2-thione hydrochloride; (R)-5-(2-aminoethyl)-1-(6-methoxychroman-3-yl)-1,3-dihydroimidazole-2-thione hydrochloride; (R)-5-(2-aminoethyl)-1-(8-methoxychroman-3-yl)-1,3-dihydroimidazole-2-thione hydrochloride; (R)-5-(2-aminoethyl)-1-(6-fluorochroman-3-yl)-1,3-dihydroimidazole-2-thione hydrochloride; (R)-5-(2-aminoethyl)-1-(8-fluorochroman-3-yl)-1,3-dihydroimidazole-2-thione hydrochloride; (R)-5-(2-aminoethyl)-1-(6,7-difluorochroman-3-yl)-1,3-dihydroimidazole-2-thione hydrochloride; (R)-5-(2-aminoethyl)-1-(6,8-difluorochroman-3-yl)-1,3-dihydroimidazole-2-thione hydrochloride; (S)-5-(2-aminoethyl)-1-(6,8-difluorochroman-3-yl)-1,3-dihydroimidazole-2-thione hydrochloride; (R)-5-(2-aminoethyl)-1-(6,7,8-trifluorochroman-3-yl)-1,3-dihydroimidazole-2-thione hydrochloride; (R)-5-(2-aminoethyl)-1-(6-chloro-8-methoxychroman-3-yl)-1,3-dihydroimidazole-2-thione hydrochloride; (R)-5-(2-aminoethyl)-1-(6-methoxy-8-chlorochroman-3-yl)-1,3-dihydroimidazole-2-thione hydrochloride; (R)-5-(2-aminoethyl)-1-(6-nitrochroman-3-yl)-1,3-dihydroimidazole-2-thione hydrochloride; (R)-5-(2-aminoethyl)-1-(8-nitrochroman-3-yl)-1,3-dihydroimidazole-2-thione hydrochloride; (R)-5-(2-aminoethyl)-1-[6-(acetylamino)chroman-3-yl]-1,3-dihydroimidazole-2-thione hydrochloride; (R)-5-aminomethyl-1-chroman-3-yl-1,3-dihydroimidazole-2-thione hydrochloride; (R)-5-aminomethyl-1-(6-hydroxychroman-3-yl)-1,3-dihydroimidazole-2-thione hydrochloride; (R)-5-(2-aminoethyl)-1-(6-hydroxy-7-benzylchroman-3-yl)-1,3-dihydroimidazole-2-thione hydrochloride; (R)-5-aminomethyl-1-(6,8-difluorochroman-3-yl)-1,3-dihydroimidazole-2-thione hydrochloride; (R)-5-(3-aminopropyl)-1-(6,8-difluorochroman-3-yl)-1,3-dihydroimidazole-2-thione hydrochloride; (S)-5-(3-aminopropyl)-1-(5,7-difluoro-1,2,3,4-tetrahydronaphthalen-2-yl)-1,3-dihydroimidazole-2-thione hydrochloride; (R,S)-5-(2-aminoethyl)-1-(6-hydroxythiochroman-3-yl)-1,3-dihydroimidazole-2-thione hydrochloride; (R,S)-5-(2-aminoethyl)-1-(6-methoxythiochroman-3-yl)-1,3-dihydroimidazole-2-thione hydrochloride; (R)-5-(2-benzylaminoethyl)-1-(6-methoxychroman-3-yl)-1,3-dihydroimidazole-2-thione hydrochloride; (R)-5-(2-benzylaminoethyl)-1-(6-hydroxychroman-3-yl)-1,3-dihydroimidazole-2-thione hydrochloride; (R)-1-(6-hydroxychroman-3-yl)-5-(2-methylaminoethyl)-1,3-dihydroimidazole-2-thione hydrochloride; (R)-1-(6,8-difluorochroman-3-yl)-5-(2-methylaminoethyl)-1,3-dihydroimidazole-2-thione hydrochloride or (R)-1-chroman-3-yl-5-(2-methylaminoethyl)-1,3-dihydroimidazole-2-thione hydrochloride.

11. A method of treating one or more of the following indications: congestive heart failure, angina, arrhythmias, circulatory disorders, Raynaud's Phenomenon, migraine, and anxiety and anxiety disorders, said method comprising the step of administering a therapeutically effective amount of a compound of formula I: 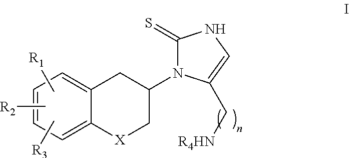
where R₁, R₂ and R₃ are the same or different and signify hydrogens, halogens, alkyl, alkylaryl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof; wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine, to a patient in need thereof.

12. The method according to claim 11, wherein the compound of formula I is (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5-(2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3-dihydroimidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzylchroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-aminoethyl)-1-(6-hydroxythiochroman-3-yl)-1,3-dihydroimidazole-2-thione; (R,S)-5-(2-aminoethyl)-1-(6-methoxythiochroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or (R)-1-chroman-3-yl-5-(2-methylaminoethyl)-1,3-dihydroimidazole-2-thione, or a pharmaceutically acceptable salt thereof.

13. The method according to claim 12, wherein the pharmaceutically acceptable salt is the hydrochloride salt.

14. A pharmaceutical composition for the treatment of one or more of the following indications: congestive heart failure, angina, arrhythmias, circulatory disorders, Raynaud's Phenomenon, migraine, and anxiety and anxiety disorders, said composition comprising a compound of formula I: 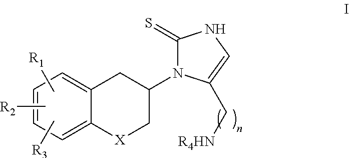
where R₁, R₂ and R₃ are the same or different and signify hydrogens, halogens, alkyl, alkylaryl, alkyloxy, hydroxy, nitro, amino, alkylcarbonylamino, alkylamino or dialkylamino group; R₄ signifies hydrogen, alkyl or alkylaryl group; X signifies CH₂, oxygen atom or sulphur atom; n is 1, 2 or 3, with the proviso that when n is 1, X is not CH₂; and the individual (R)- and (S)-enantiomers or mixtures of enantiomers and pharmaceutically acceptable salts thereof; wherein the term alkyl means hydrocarbon chains, straight or branched, containing from one to six carbon atoms, optionally substituted by aryl, alkoxy, halogen, alkoxycarbonyl or hydroxycarbonyl groups; the term aryl means a phenyl or naphthyl group, optionally substituted by alkyloxy, halogen or nitro group; the term halogen means fluorine, chlorine, bromine or iodine.

15. The pharmaceutical composition according to claim 14, wherein the compound of formula I is (S)-5-(2-aminoethyl)-1-(1,2,3,4-tetrahydronaphthalen-2-yl)-1,3-dihydroimidazole-2-thione; (S)-5-(2-aminoethyl)-1-(5,7-difluoro-1,2,3,4-tetrahydronaphthalen-2-yl)-1,3-dihydroimidazole-2-thione; (R)-5-(2-aminoethyl)-1-chroman-3-yl-1,3-dihydroimidazole-2-thione; (R)-5-(2-aminoethyl)-1-(6-hydroxychroman-3-yl)-1,3-dihydroimidazole-2-thione; (R)-5-(2-aminoethyl)-1-(8-hydroxychroman-3-yl)-1,3-dihydroimidazole-2-thione; (R)-5-(2-aminoethyl)-1-(6-methoxychroman-3-yl)-1,3-dihydroimidazole-2-thione; (R)-5-(2-aminoethyl)-1-(8-methoxychroman-3-yl)-1,3-dihydroimidazole-2-thione; (R)-5-(2-aminoethyl)-1-(6-fluorochroman-3-yl)-1,3-dihydroimidazole-2-thione; (R)-5-(2-aminoethyl)-1-(8-fluorochroman-3-yl)-1,3-dihydroimidazole-2-thione; (R)-5-(2-aminoethyl)-1-(6,7-difluorochroman-3-yl)-1,3-dihydroimidazole-2-thione; (R)-5-(2-aminoethyl)-1-(6,8-difluorochroman-3-yl)-1,3-dihydroimidazole-2-thione; (S)-5-(2-aminoethyl)-1-(6,8-difluorochroman-3-yl)-1,3-dihydroimidazole-2-thione; (R)-5-(2-aminoethyl)-1-(6,7,8-trifluorochroman-3-yl)-1,3-dihydroimidazole-2-thione; (R)-5-(2-aminoethyl)-1-(6-chloro-8-methoxychroman-3-yl)-1,3-dihydroimidazole-2-thione; (R)-5-(2-aminoethyl)-1-(6-methoxy-8-chlorochroman-3-yl)-1,3-dihydroimidazole-2-thione; (R)-5-(2-aminoethyl)-1-(6-nitrochroman-3-yl)-1,3-dihydroimidazole-2-thione; (R)-5-(2-aminoethyl)-1-(8-nitrochroman-3-yl)-1,3-dihydroimidazole-2-thione; (R)-5-(2-aminoethyl)-1-[6-(acetylamino)chroman-3-yl]-1,3-dihydroimidazole-2-thione; (R)-5-aminomethyl-1-chroman-3-yl-1,3-dihydroimidazole-2-thione; (R)-5-aminomethyl-1-(6-hydroxychroman-3-yl)-1,3-dihydroimidazole-2-thione; (R)-5-(2-aminoethyl)-1-(6-hydroxy-7-benzyl chroman-3-yl)-1,3-dihydroimidazole-2-thione; (R)-5-aminomethyl-1-(6,8-difluorochroman-3-yl)-1,3-dihydroimidazole-2-thione; (R)-5-(3-aminopropyl)-1-(6,8-difluorochroman-3-yl)-1,3-dihydroimidazole-2-thione; (S)-5-(3-aminopropyl)-1-(5,7-difluoro-1,2,3,4-tetrahydronaphthalen-2-yl)-1,3-dihydroimidazole-2-thione; (R,S)-5-(2-aminoethyl)-1-(6-hydroxythiochroman-3-yl)-1,3-dihydroimidazole-2-thione; (R,S)-5-(2-aminoethyl)-1-(6-methoxythio chroman-3-yl)-1,3-dihydroimidazole-2-thione; (R)-5-(2-benzylaminoethyl)-1-(6-methoxychroman-3-yl)-1,3-dihydroimidazole-2-thione; (R)-5-(2-benzylaminoethyl)-1-(6-hydroxychroman-3-yl)-1,3-dihydroimidazole-2-thione; (R)-1-(6-hydroxychroman-3-yl)-5-(2-methylaminoethyl)-1,3-dihydroimidazole-2-thione; (R)-1-(6,8-difluorochroman-3-yl)-5-(2-methylaminoethyl)-1,3-dihydroimidazole-2-thione or (R)-1-chroman-3-yl-5-(2-methylaminoethyl)-1,3-dihydroimidazole-2-thione, or a pharmaceutically acceptable salt thereof.

16. The pharmaceutical composition according to claim 15, wherein the pharmaceutically acceptable salt is the hydrochloride salt.
